(19) United States
(12) Patent Application Publication
(10) Pub. No.: US 2009/0137945 A1
(43) Pub. Date: May 28, 2009

(54) Electro Collagen Induction Therapy Device

(76) Inventor: Claire Marquez, Santa Fe, NM (US)

Correspondence Address: QUICKPATENTS, INC., 32861 CALLE PERFECTO, SUITE A, SAN JUAN CAPISTRANO, CA, 92675, US

(21) Appl. No.: 12/277,845

(22) Filed: Nov. 25, 2008

Related U.S. Application Data
(60) Provisional application No. 61/004,728, filed on Nov. 28, 2007.

(51) Int. Cl. A61B 17/34; A61M 5/32

(52) U.S. Cl. 604/46; 606/186

(57) ABSTRACT

A device for creating a plurality of small wounds in a patient's skin and introducing topically-applied substances therein is disclosed. The device comprises a rigid plate that has a top side, a bottom side, and at least one peripheral edge that connects the top and bottom sides. The device further includes a tool attachment means fixed with the top side of the plate and adapted to fix the device to a motorized tool that oscillates reciprocally back and forth along the longitudinal axis of the plate. A plurality of needles project generally orthogonally away from the bottom side of the plate. In use, the plate is fixed with the tool and the tool activated. The bottom side of the plate is then applied to the skin of the patient such that each needle repeatedly pierces same to create the plurality of small wounds therein. When the substance is applied to the skin first, each needle draws a portion of the substance into the skin with each oscillation of the tool.

### CROSS-REFERENCE TO RELATED APPLICATIONS

This application claims the benefit of US Provisional Patent Application 61/004,728, filed on Nov. 28, 2007, and incorporated herein by reference.

### STATEMENT REGARDING FEDERALLY SPONSORED RESEARCH AND DEVELOPMENT

Not Applicable.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### FIELD OF THE INVENTION

This invention relates to collagen induction therapy, and more particularly to a device and method for delivering same.

### DISCUSSION OF RELATED ART

Collagen induction therapy is well known to provide beneficial effects to a patient's skin, such as remodeling of pitted acne scarring, surgical and trauma scarring, burn contractures, stretch marks, and the like. Deep facial lines such as “crow's feet” may also be treated with such therapy, which increases elasticity and melanocytes in the patient's skin. Body temperature, blood flow and human growth hormone can also be increased with such therapy, in addition to other benefits.

Conventional collagen induction therapy is performed by providing a roller having a plurality of relatively thin radially-aligned needles that are just long enough to pierce the patient's epidermis layer. Such a device is disclosed in US Patent Application 2007/0073217 to James on Mar. 29, 2007. Such rolling prior art devices have the drawback that the needles are inserted into the epidermis at a slight angle as the device is rolled along the skin, and then removed at a slight opposing angle. As such, each needle slightly tears the skin instead of piercing it, which results in increased and undesired trauma to the patient. Further, the needles of such devices tend to become more malleable and bend with repeated use, particularly as such needles become warmer through contact with the patient's skin. Further, small areas of the skin, such as around the patient's eyes, are difficult to treat with such a rolling device since there may not be enough room to roll such a device in the desired treatment area.

Other prior art devices have a flat bed of small needles, which are typically hand-held for manual administration thereof to the skin, such as disclosed in US Patent Application 2007/0118073 to Martin et al. on May 24, 2007, and US Patent Application 2007/0049901 to Wu et al. on Mar. 1, 2007. Manual devices of this type are not able to practically deliver thousands of microperforations to the skin within a short period of time, such as several minutes. Further, such manually applied devices have the drawback that they are not easily aligned orthogonally to the patient's skin, and thus such devices and methods share many of the same drawbacks with the roller-type devices previously described.

Therefore, there is a need for a device that delivers collagen induction therapy quickly and easily to a patient's skin. Such a needed device would be capable of piercing the patient's skin with many thousands of microperforations within a short period of time, and the needles of such a device would remain straight, and therefore, sharp, for the duration of their use. Such a needed device would be readily used in conjunction with a topically-applied substance for introduction into the skin of the patient, and would allow for easy cleaning and sterilizing of the needles of such a device. The needed device would allow for different needle quantity, thicknesses and lengths to be readily interchanged and used as desired. Such a needed invention would also be relatively easy to use and reduce manual fatigue in the operator of such a device. The needed device would be relatively inexpensive to manufacture. The present invention accomplishes these objectives.

### SUMMARY OF THE INVENTION

The present device is a device for creating a plurality of small wounds in the skin of a patient by utilizing a motorized tool that oscillates back and forth thousands of times per minute, such as a digital permanent cosmetic marking tool. The device comprises a rigid plate that has a top side, a bottom side, and at least one peripheral edge that connects the top and bottom sides. The device further includes a tool attachment means fixed with the top side of the plate and adapted to fix the device to the motorized tool. A plurality of needles project generally orthogonally away from the bottom side of the plate.

In use, the plate is fixed with the tool and the tool activated to oscillate the plate back and forth along its longitudinal axis. The bottom side of the plate is then applied to the skin of the patient such that each needle repeatedly pierces the skin of the patient to create the plurality of small wounds therein. When a substance is applied to the skin first, each needle draws a portion of the substance into the skin with each oscillation of the tool. Such a topically-applied substance may be, for example, a topical anesthetic, a vascular constrictor to inhibit bleeding and swelling, or the like.

The present invention is a device that can delivers collagen induction therapy quickly and easily to a patient's skin. The present device is capable of piercing the patient's skin with many thousands of microperforations within a short period of time, and the needles of such a device remain straight and sharp for the duration of their use. The present invention is readily used in conjunction with a topically-applied substance for introduction into the skin of the patient, allows for easy cleaning and sterilizing of the needles of such a device, and allows for different needle quantities, thicknesses and lengths to be readily interchanged and used as desired. The present device is also relatively easy to use and reduces manual fatigue of the operator of such a device. The present invention is relatively inexpensive to manufacture. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the invention as fixed to a motorized tool;

FIG. 2 is a side elevational view of the invention, illustrated as detached from the motorized tool;

FIG. 3 is an enlarged front elevational view of the invention as applied to the skin of a patient;

FIG. 4 is an enlarged portion of the invention as taken along lines 4-4 of FIG. 3;

FIG. 5 is a bottom plan view of an oval-shaped plate of the invention; and

FIG. 6 is a bottom plan view of a rectangular-shaped plate of the invention.

### DETAILED DESCRIPTION OF THE PREFERRED EMBODIMENT

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

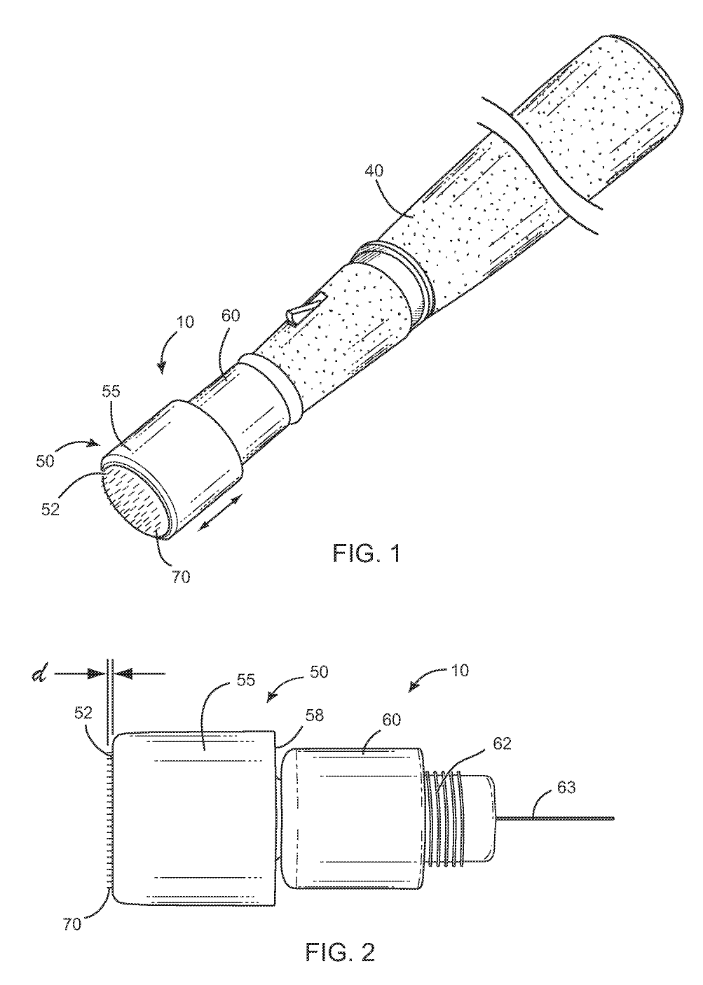

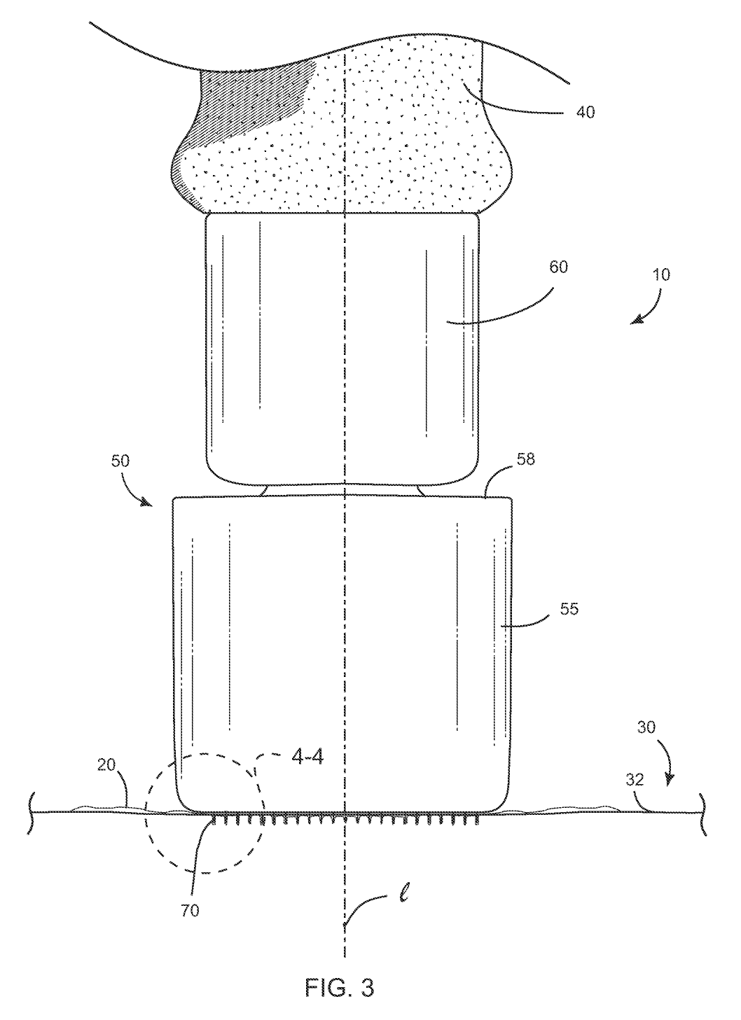

FIGS. 1, 3 and 4 illustrate a device for creating a plurality of small wounds 22 in the skin 32 of a patient 30 by utilizing a motorized tool 40 that oscillates back and forth thousands of times per minute, such as a permanent cosmetic marking tool. An example of such a tool is produced by Shenzhen Jianlemei Cosmetic Co., Ltd, of China, under the Kanagawa Brand as the Kanagawa Digital Control Permanent Make Up Machine, which has several speed settings such as 13,000 cycles-per-minute (CPM), 15,000 CPM, and 18,800 CPM, for example.

The device 10 comprises a rigid plate 50 that has a top side 58, a bottom side 52, and at least one peripheral edge 55 that connects the top and bottom sides 58,52. The plate 50 has a longitudinal axis l substantially orthogonal to the bottom side 52 of the plate 50, and preferably also orthogonal to the top side 58 of the plate 50. The plate 50 is preferably made from a cold sterilizable rigid plastic material, but can also be made from any other suitable rigid plastic, ceramic, or metal material.

Preferably the plate 50 is a cylinder, as illustrated in FIGS. 1-4. However, the plate 50 may also be an oval in plan view (FIG. 5), a rectangle in plan view (FIG. 6), or any other suitable shape. In one embodiment wherein the plate 50 is a cylinder, the plate 50 is between 0.4 and 0.5 inches in diameter. In another embodiment wherein the plate 50 is a cylinder, the plate 50 is between 0.9 and 1.1 inches in diameter. In another embodiment wherein the plate 50 is a cylinder, the plate 50 is between 1.9 and 2.1 inches in diameter.

The device 10 further includes a tool attachment means 60 fixed with the top side 58 of the plate 50 and adapted to fix the device 10 to the motorized tool 40. Such a tool attachment means 60 may include a threaded shaft 62 (FIG. 2), for example, or any other suitable mechanical fastener for adapting the device 10 to the tool 40. A connecting rod 63 may be included for facilitating a rigid connection between the device 10 and an oscillating member (not shown) of the tool 40.

A plurality of needles 70, such as several dozen, for example, project generally orthogonally away from the bottom side 52 of the plate 50 by a distance d (FIGS. 1 and 2). Such a plurality of needles 70 may also be known as a micro projection array. Preferably each needle is made from medical-grade steel, such as an acupuncture-type needle, and is between 0.1 and 0.5 mm in diameter, and preferably close to 0.25 mm. Preferably each needle is between 0.4 and 2.1 mm in length. In one embodiment of the invention, the length of each needle 70 is substantially 1.0 mm in length, being between 0.5 mm and 1.1 mm. In another embodiment of the invention, the length of each needle 70 is substantially 1.5 mm in length, between 1.4 mm and 1.6 mm. In another embodiment of the invention, the length of each needle 70 is substantially 2.0 mm in length, between 1.9 mm and 2.5 mm.

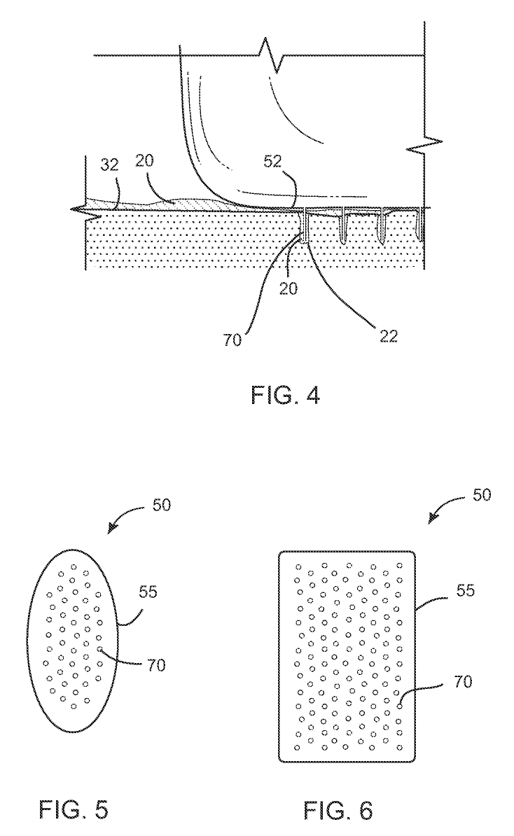

In use, the plate 50 may be fixed with the tool 40 and the tool activated to oscillate the plate back and forth along its longitudinal axis l. The bottom side 52 of the plate 50 is then applied to the skin 32 of the patient 30 such that each needle 70 repeatedly pierces the skin 32 of the patient 30 to create the plurality of small wounds 22 therein (FIG. 4). When a substance 20 is applied to the skin 32 first, each needle 70 draws a portion of the substance 20 into the skin 32 with each oscillation of the tool 40. Such a topically-applied substance 20 may be, for example, a topical anesthetic, a vascular constrictor to inhibit bleeding and swelling, or the like.

While a particular form of the invention has been illustrated and described, it will be apparent that various modifications can be made without departing from the spirit and scope of the invention. For example, various types of tools 40 may be used with the device 10, provided that the tool 40 oscillates in a reciprocal motion generally parallel to the longitudinal axis l of the plate 50. Accordingly, it is not intended that the invention be limited, except as by the appended claims.

The teachings provided herein can be applied to other systems, not necessarily the system described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above Detailed Description. While the above description details certain embodiments of the invention and describes the best mode contemplated, no matter how detailed the above appears in text, the invention can be practiced in many ways. Details of the system may vary considerably in its implementation details, while still being encompassed by the invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A device for creating a plurality of small wounds in the skin of a patient with a motorized tool, comprising:
a rigid plate having a top side, a bottom side, and at least one peripheral edge connecting the top and bottom sides, the plate having a longitudinal axis substantially orthogonal to the bottom side;
a tool attachment means fixed with the top side of the plate, the tool attachment means adapted to fix the device to the motorized tool; and
a plurality of needles each projecting generally orthogonally to the bottom side of the plate a predetermined distance;
whereby the plate may be fixed with the tool and the tool activated to oscillate the plate back and forth along its longitudinal axis, the bottom side of the plate applied to the skin of the patient, each needle piercing the skin of the patient to create the plurality of small wounds therein.

2. The device of claim 1 wherein each needle is between 0.5 and 0.1 mm in diameter.

3. The device of claim 1 wherein each needle is between 0.5 and 1.1 mm in length.

4. The device of claim 1 wherein each needle is between 1.4 and 1.6 mm in length.

5. The device of claim 1 wherein each needle is between 1.9 and 2.5 mm in length.

6. The device of claim 1 wherein the plate is a cylinder.

7. The device of claim 6 wherein the diameter of the plate is between 0.4 and 0.5 inches.

8. The device of claim 6 wherein the diameter of the plate is between 0.9 and 1.1 inches.

9. The device of claim 6 wherein the diameter of the plate is between 1.9 and 2.1 inches.

10. The device of claim 1 wherein the plate is oval in the top plan view thereof.

11. The plate of claim 1 wherein the plate is rectangular in the top plan view thereof.

12. A method of creating a plurality of small wounds in the skin of a patient with a motorized tool, the method comprising the steps of:
a) providing a device as recited in claim 1;
b) attaching the device to the motorized tool;
c) activating the motorized tool; and
d) applying the device to the skin of the patient to create the plurality of small wounds in the skin of the patient.

13. A method of introducing a substance into the skin of a patient with a motorized tool, the method comprising the steps of:
a) providing a device as recited in claim 1;
b) attaching the device to the motorized tool;
c) applying the substance to the skin of the patient;
d) activating the motorized tool; and
e) applying the device to the skin of the patient to introduce the substance into the skin of the patient.

14. The method of claim 13 wherein step e) is applying the device to the skin of the patient at an angle substantially orthogonal to the longitudinal axis of the plate, to introduce the substance into the skin of the patient.
